(19) United States
(12) Patent Application Publication
(10) Pub. No.: US 2009/0047352 A1
(43) Pub. Date: Feb. 19, 2009

(54) Colourant Compositions and Their Use

(76) Inventors: Michael Francis Butler, Sharnbrook (GB); Ramin Djalali, Sharnbrook (GB); Krassimir Petkov Velikov, Vlaardingen (NL)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/886,371

(22) PCT Filed: Mar. 13, 2006
(86) PCT No.: PCT/EP2006/002500

§ 371 Date: Sep. 14, 2007

(30) Foreign Application Priority Data
Mar. 16, 2005 (EP) 05251575.6

(51) Int. Cl. A61K 8/02; D06M 11/00; C11D 11/00

(52) U.S. Cl. 424/489; 424/70.6; 8/115.51; 106/31.13

(57) ABSTRACT

A fibre colourant and an ink composition are provided which comprise monodisperse particles capable of forming a colloidal crystal that diffracts light having a wavelength in a range that corresponds to the wavelength of visible light. The use of such compositions in colouring substrates is also provided.

### FIELD OF THE INVENTION

The present invention relates to fibre colourant composition and ink compositions comprising monodisperse particles.

### BACKGROUND OF THE INVENTION

WO-A-2005/063902 discloses ink jet compositions that comprise from about 0.5 to about 70%, preferably from about 1 to about 50%, more preferably from about 1 to about 30%, particularly from about 5 to about 20%, by weight of monodisperse particles. The monodisperse particles form three-dimensional photonic crystals on a substrate after application to the substrate surface by arranging in a three-dimensional, tightly packed, regular and spherical structure on the substrate surface. One example contains 10% by weight of the monodisperse particles (i.e. polymethylmethacrylate solids) and the carrier is a mixture of water and dipropylene glycol methyl ether.

US-A-2004/0234746 teaches that crystallisation of monodisperse particles may occur by spraying an aqueous suspension of the monodisperse particles directly onto a substrate with subsequent drying. In this case, the composition comprises from 5 to 20% by weight of the monodisperse particles. According to one example, crystallisation is achieved by drying of a dispersion of monodisperse particles on a horizontal substrate. The crystals so formed are used to prepare coating compositions. In another example, crystallisation is achieved directly on the substrate to be coated, i.e. by spraying the suspension onto the substrate followed by drying. The suspension is a 5 to 20% aqueous suspension.

US-A-2003/0125416 discloses colourants that include an ordered array of particles held in a polymeric matrix. The process for preparing the array includes the steps of providing an aqueous dispersion of particles in a carrier, applying the dispersion onto a substrate and evaporating the carrier to provide an ordered periodic array of particles on the substrate. The dispersion may comprise from about 1 to about 70%, preferably from about 30 to about 65%, by volume of the particles.

US-A-2003/0008771 teaches that upon application of a suspension of monodisperse spheres and colloidal species to a flat surface the spheres crystallise into closely packed layers with the evaporation of the solvent. It is disclosed that the preferred concentration of a silica suspension is from about 5 to about 65%, preferably from about 20 to about 50%, by weight and preferably from about 40 to about 50% by weight for a moving substrate process. In the examples, a silica sphere suspension (having a concentration of 11% by weight) is used, along with a silica sol (having a concentration of 40 to 41% by weight) or a tin (IV) oxide sol (having a concentration of 15% by weight). The examples use anhydrous ethanol as the solvent, which is evaporated off at room temperature overnight.

US-A-2003/0116062 describes pigments that have a three-dimensional periodic arrangement of monodisperse spheres in the nanometer range and a process for preparing the pigments by applying a suspension of the monodisperse particles to a substrate and removing the liquid medium. The suspension may comprise from 1 to 35% by weight of the monodisperse spheres. The examples use ethanol as the solvent.

U.S. Pat. No. 6,337,131 discloses colourants that have at least domains of regularly arranged cores of core-shell particles. The particles form a regular, crystal lattice type array upon application to a surface.

We have now discovered that colloidal crystals may be used as colourants on hair or fabrics or as inks by in situ formation on the substrate in question.

### DEFINITION OF THE INVENTION

In a first aspect, the present invention provides a method of colouring a substrate selected from hair of an individual and fabric fibres, which method comprises contacting the substrate with a composition comprising monodisperse particles capable of forming a colloidal crystal that diffracts light having a wavelength in a range that corresponds to the wavelength of visible light, such that colloidal crystals comprising the monodisperse particles form on the substrate.

Preferably the colloidal crystal has a lattice spacing in a range that corresponds to the wavelength of visible light.

In one embodiment, the particles are inorganic. In an alternative embodiment, the particles are organic polymers.

In one embodiment, the fibre colourant composition is a hair colourant composition. In another embodiment the fibre colourant composition is a textile colourant composition. In a further embodiment, the fibre colourant composition is an ink composition, i.e. suitable for printing onto a printable surface such as paper or fabrics.

In a related aspect the present invention provides a method of printing onto a substrate which method comprises contacting at least a region of the substrate with an ink composition comprising monodisperse particles capable of forming a colloidal crystal having a lattice spacing in a range that corresponds to the wavelength of visible light, such that colloidal crystals comprising the monodisperse particles form on at least a portion of the substrate.

In a related aspect the present invention provides use of a colourant composition comprising monodisperse particles capable of forming a colloidal crystal that diffracts light having a wavelength in a range that corresponds to the wavelength of visible light, in the manufacture of a product for colouring the hair of an individual.

Similarly, the invention provides use of a fibre colourant composition comprising monodisperse particles capable of forming a colloidal crystal that diffracts light having a wavelength in a range that corresponds to the wavelength of visible light, in the manufacture of a product for colouring the fibres in a fabric.

Yet another aspect of the invention provides use of a colourant composition comprising monodisperse particles capable of forming a colloidal crystal that diffracts light having a wavelength in a range that corresponds to the wavelength of visible light, in the manufacture of an ink.

The present invention also provides a fibrous material comprising, typically thereon or within, at least one colloidal crystalline layer comprising monodisperse particles, which layer diffracts light having a wavelength in a range that corresponds to the wavelength of visible light. In one embodiment, the fibrous material is a fabric. Preferably, the fibrous material comprises at least two or three layers of the colloidal crystals.

In another aspect, the present invention provides an ink composition comprising monodisperse particles capable of forming a colloidal crystal that diffracts light having a wavelength in a range that corresponds to the wavelength of visible light.

The present invention also provides a method of printing onto a substrate which method comprises contacting at least a region of the substrate with an ink composition of the invention such that colloidal crystals comprising the monodisperse particles and that diffract light having a wavelength in a range that corresponds to the wavelength of visible light form on at least a portion of the substrate. Preferably the substrate is paper or fabric.

Preferably, the ink composition is applied to form letters, numbers or other symbols, or a graphic design on the substrate.

The present invention also provides a substrate onto which has been applied an ink composition of the invention to form letters, numbers or other symbols, or a graphic design on the substrate. Preferably the substrate is paper or fabric.

In a related aspect the present invention provides a substrate which comprises, typically thereon or within, at least one colloidal crystalline layer comprising monodisperse particles, which layer diffracts light having a wavelength in a range that corresponds to the wavelength of visible light, the crystalline layer forming letters, numbers or other symbols, or a graphic design on the substrate. Preferably the substrate is a fibrous substrate such as of paper or fabric.

In the various aspects and embodiments described above, it is preferred that the lattice spacing in at least one axis is from about 350 nm to about 770 nm. In the various aspects and embodiments described above, it is preferred that the particles are spherical.

Our co-pending European Patent Application No. 05257136, unpublished at the filing date of the present application, describes and claims colourant composition comprising (i) monodisperse particles capable of forming a colloidal crystal and (ii) at least one broad spectrum absorber contrast agent. A broad spectrum absorber contrast agent causes a narrowing of the spectral peak of the absorbed colour and therefore an enhancement of the structural colourant effect. One class of colourant or ink compositions utilised in the present invention is substantially devoid of such broad spectrum absorber contrast agent(s).

### DETAILED DESCRIPTION OF THE INVENTION

Unless defined otherwise, all technical and scientific terms used herein have the same meaning as commonly understood by one of ordinary skill in the art.

The terms “colour” and “coloured” as used herein include “white”, and colouring of substrates and fibres includes “brightening”, for example the brightening of textiles.

### Monodisperse Particles

The fibre colourant compositions or ink compositions of the invention comprise monodisperse particles capable of forming a colloidal crystal that appears coloured to the human eye.

Monodisperse particles are defined as having at least 60% of the particles fall within a specified particle size range. Monodispersed particles deviate less than 10% in root mean square (rms) diameter. Highly monodisperse particles deviate less than 5% in rms diameter. Monodisperse particles for use in the invention typically have an rms diameter of less than about 1 μm and greater than about 1 nm, and are therefore classed as nanoparticles. Preferably the monodisperse particles have an rms diameter of greater than about 150 or about 200 nm. Preferably the monodisperse particles have an rms diameter of less than about 900 nm or about 800 nm. More preferably the diameter of the monodisperse particles is from about 200 nm to about 550 nm.

The monodisperse particles are chosen such that they can form a colloidal crystal which appears coloured to the human eye, i.e. in the visible spectrum. The crystal colour or colours observed depend principally on two factors, namely the lattice spacing within the colloidal crystal and the refractive index of the particles and matrix, which affects the wavelength of light diffracted. The lattice spacing is determined by factors such as the size of the monodisperse particle. For example, we have used particles having a diameter of from 250 to 510 nm to generate coloured colloidal crystals having colours ranging from blue and red to green and yellow. Colloidal crystals can have different colours when viewed from different angles because the lattice spacing can be different in different axes of the crystal. Provided that the lattice spacing in at least one axis results in diffraction of light with a wavelength in the visible spectrum then the crystal will appear to be coloured.

Preferably, the lattice spacing in at least one axis is from about 350 to about 780 nm, preferably from 380 to 770 nm.

Monodisperse particles can be of varying geometry. In a preferred embodiment, the monodisperse particles are substantially spherical.

The monodisperse particles suitable for use in the colourant compositions of the present invention may be made from any suitable material, including one or more selected from organic and/or inorganic materials. For example, suitable organic materials include organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles. Suitable inorganic materials include metal chalcogenide, metal pnictide, silica, metal and metal oxide particles. Examples of suitable metal oxides include, for example, Al₂O₃, TiO₂, SnO₂, Sb₂O₅, Fe₂O₃, ZrO₂, CeO₂ and Y₂O₃. Examples of suitable metals include, for example, gold, copper and silver.

By the term “metal chalcogenide” we mean metal compounds formed with anions from group 16 of the Periodic Table of Elements (according to established IUPAC nomenclature), i.e. oxygen, sulphur, selenium, tellurium and polonium.

By the term “metal pnictide” we mean metal compounds formed with anions from group 15 of the Periodic Table of Elements (according to established IUPAC nomenclature), i.e. nitrogen, phosphorus, arsenic, antimony and bismuth.

Monodispersed poly(methylmethacrylate) composites may be prepared following the process described by M. Egen, R. Zentel (Macromol. Chem. Phys. 2004, 205, 1479-1488) or are commercially available from Duke Scientific Corporation.

Methods for preparing monodisperse particles are known in the art. Dispersions may be prepared using emulsion, dispersion, suspension polymerization if particles are polymeric, or if particles are inorganic (e.g., silica particles) the dispersion may be prepared using sol-gel processes.

Monodispersed silica spheres can be prepared following the well-known process by Stöber, Fink and Bohn (J. Colloid Interface Sci. 1968, 26,62). The process was later refined by Bogush, et. al. (J. Non-Crys. Solids 1988, 104, 95). Alternatively, silica particles can be purchased from Blue Helix, Limited or they can be freshly prepared by the process described in U.S. Pat. No. 4,775,520 and U.S. Pat. No. 4,911,903.

For example, monodisperse silica spheres can be produced by hydrolytic polycondensation of tetraalkoxysilanes in an aqueous-ammoniacal medium, a sol of primary particles being produced first of all and then the silica particles obtained being brought to the desired particle size by continuous, controlled addition of tetraalkoxysilane. With this process it is possible to produce monodisperse SiO₂ spheres having average particle diameters of between 0.05 and 10 μm with a standard deviation of less than 7%.

U.S. Pat. No. 6,800,709 describes preparation of monodisperse particles with a narrow size distribution by free radical polymerization or copolymerization of hydrophobic monomers in a water-based system in the presence of cyclodextrin. Suitable hydrophobic monomers include styrenics, acrylonitrile, methacrylonitrile, acrylates, methacrylates, methacryl amides, acrylamides, maleimides; vinyl ethers, vinyl esters, monoalkylmaleates, dialkyl maleates, fluorinated acrylates and fluorinated methacrylates.

The monodisperse particles can be combined with suitable carriers and/or other components, such as solvents, to form compositions of the invention, such as fibre colourant compositions or ink compositions. Compositions can typically be in liquid form; semi-liquid form including lotions, pastes, creams; or solid form including powders e.g. laundry powders or tablets. The amount of monodisperse particles present in such compositions is typically from about 0.1 wt % to about 10 wt % in liquid and semi-liquid compositions and from about 1 wt % to about 40 wt % in solid compositions. Ink compositions will typically comprise from about 4 wt % to about 50 wt % of the monodisperse particles. The maximum amount of such monodisperse particles in any composition could even be as low as less than 5 wt % or less than 1 wt % or even less than 0.5 wt %.

### Colloidal Crystals

The colourant compositions of one aspect of the present invention comprise monodisperse particles capable of forming a colloidal crystal, for example upon application of the colourant composition to a substrate.

For the avoidance of doubt, references herein to “a colloidal crystal” are intended to relate to one or more colloidal crystals.

By the term “colloidal crystal” we mean a regular array of monodisperse particles having a substantially regular or constant spacing therebetween. Thus, the array of monodisperse particles forms a dispersed phase arranged in a continuous phase (or matrix). The continuous phase (or matrix) may comprise a gas, a liquid or a solid of a different refractive index to the dispersed phase.

As the skilled person would appreciate, a colloidal crystal may, however, contain some impurities and/or defects. The levels of impurities and/or defects typically will depend on the materials and methods of preparation used.

The term “colloidal crystal” has the same meaning as the term “super-lattice”. A colloidal crystal or super-lattice is a type of photonic crystal, which is an optical, artificial structure characterised by 2D or 3D periodic arrangements of dielectric material which lead to the formation of energy band structures for electromagnetic waves propagating them.

### Fibres

A fibre is a fine hair-like structure of biological, mineral or synthetic origin. In the context of the present invention, fibres include animal or human hair. The fibres may be part of a fabric, such as a textile or nonwoven fabric.

Commercially available fibres have diameters ranging from less than about 0.001 mm to more than about 0.2 mm and they come in several different forms: short fibres (known as staple, or chopped), continuous single fibres (filaments or monofilaments), untwisted bundles of continuous filaments (tow), and twisted bundles of continuous filaments (yarn). Fibres are classified according to their origin, chemical structure, or both. They can be braided into ropes and cordage, made into felts (also called nonwovens or nonwoven fabrics), woven or knitted into textile fabrics, or, in the case of high-strength fibres, used as reinforcements in composites.

Fibres may be natural fibres, synthetic or man-made fibres, or combinations thereof. Examples of natural fibres include but are not limited to: animal fibres such as wool, silk, fur, and hair; vegetable fibres such as cellulose, cotton, flax, linen, and hemp; and certain naturally occurring mineral fibres. Synthetic fibres can be derived from natural fibres or not. Examples of synthetic fibres which are derived from natural fibres include but are not limited to rayon and lyocell, both of which are derived from cellulose, a natural polysaccharide fibre. Synthetic fibres which are not derived from natural fibres can be derived from other natural sources or from mineral sources. Examples of synthetic fibres derived from natural sources include polysaccharides such as starch. Examples of fibres from mineral sources include but are not limited to polyolefin fibres such as polypropylene and polyethylene fibres, which are derived from petroleum, and silicate fibres such as glass and asbestos. Synthetic fibres are commonly formed, when possible, by fluid handling processes (e.g., extruding, drawing, or spinning a fluid such as a resin or a solution). Synthetic fibres are also formed by solid handling size reduction processes (e.g., mechanical chopping or cutting of a larger object such as a monolith, a film, or a fabric).

Common synthetic fibres include but are not limited to nylon (polyamide), acrylic (polyacrylonitrile), aramid (aromatic polyamide), polyolefin (polyethylene and polypropylene), polyester and butadiene-stryene block copolymers.

### Other Substrates

Ink compositions of the invention can be applied to any suitable substrate. Preferred substrates are those with surface irregularities that act as sites for crystal nucleation, such as fibrous materials. Substrates include paper, fabrics, wood and plastics.

### Uses

Fibre colourant compositions of the invention can be used to colour the fibres in a fabric. Colouring of fibres also includes the ‘brightening’ of fibres, such in the case of white textile materials.

Fibres can be coloured by contacting the fibres, such as the hair of an individual or fabric fibres, with a composition of the invention. Hair colourant compositions are typically in the form of sprays, lotions, shampoos, creams or pastes which can be applied directly to all or part of the hair. Following a suitable contact time, excess composition can then be washed off if necessary. Preferably the composition is in contact with the hair for sufficient time such that at least two or three layers of colloidal crystals are formed.

Fibre colourant compositions for use in colouring or brightening fabrics/textiles can be applied as part of standard laundry formulations known in the art such as powders or tablets that dissolve/disperse in water or as liquids.

Ink compositions of the invention can be applied to substrates using standard printing techniques known in the art for applying inks to a range of substrates. Typically, the ink compositions are applied the substrate to form letters, numerals or other symbols, or graphic designs.

In the above applications, it is sufficient for a single layer of colloidal crystals to form on or within the substrate or fibre. However, it is preferred that at least two or three layers of colloidal crystals are formed. The coverage of colloidal crystalline layers need not be complete i.e. it can be a discontinuous layer. Depending on the substrate, which may be porous, colloidal crystals may form on the surface of, and/or within, the substrate. Further, the crystalline layer or layers need not be entirely regular, provided that the desired colour effects are achieved. In other words some crystal disorder is permitted.

### BRIEF DESCRIPTION OF THE DRAWINGS

The present invention will now be described further with reference to the following examples which are illustrative only and non-limiting, and with reference to the accompanying drawings, in which:—

FIG. 1 shows bundles of crystalline hair fibres which are coloured in accordance with the present invention, showing respective different colours;

FIG. 2 shows SEM images illustrating formation of crystalline layers on hair, in accordance with the present invention;

FIG. 3 shows images of crystalline cotton samples coloured in accordance with the present invention;

FIGS. 4 and 5 show SEM images of individual cotton fibres showing crystalline layer formation; and

FIG. 6 shows another SEM image of a fibre where crystal growth is initiated along the surface ridges of the cellulose fibre.

### EXAMPLES

### Example 1

### Assembly of Colloidal Crystals on Hair Fibres

### Synthesis of Silica-Particles

Monodispersed silica spheres were prepared following the well-known process by Stöber, Fink and Bohn (J. Colloid Interface Sci. 1968, 26,62), as refined by Bogush, et. al. (J. Non-Crys. Solids 1988, 104, 95).

Briefly, the spheres were produced by hydrolytic polycondensation of tetraalkoxysilanes in an aqueous-ammoniacal medium, a sol of primary particles being produced first of all and then the SiO₂ particles obtained being brought to the desired particle size by continuous, controlled addition of tetraalkoxysilane (see U.S. Pat. No. 4,775,520). The final particle size obtained depends on the quantity of tetraalkoxysilane added in total. With this process it is possible to produce monodisperse SiO₂ spheres having average particle diameters of between 0.05 and 10 μm with a standard deviation of less than 7%. This procedure was used to prepare monodisperse silica spheres have average particle diameters of 250 nm, 330 nm, 410 nm or 500 nm.

The samples were then purified using the following method. The dispersion was centrifuged at 3000 rpm for 20 minutes to separate the solid from the liquid. The solid was redispersed in anhydrous ethanol to the original volume by mechanical stirring and ultrasonic treatment. This procedure was repeated several times.

The dispersion so prepared was divided into 4 equal parts and each was added to a 2 ml plastic vial having a flat bottom.

### Crystal Growth on Hair Substrate

The sample consisted of a silica concentration of about 0.2 weight % in anhydrous ethanol. Switches of Caucasian hair were placed vertically into each dispersion. The dispersion in the container was left to evaporate/crystallize at room temperature overnight.

### Results

(1) For particles with sizes of approximately 250 nm, the hair-fibres showed strong bluish diffraction at a viewing angle close to the normal axis of the crystalline surface on the hair-fibres (and a reddish colour at an angle far away from the normal axis).

(2) For particles with sizes of approximately 330 nm, the hair fibres showed a strong turquoise diffraction colour at a viewing angle close to normal axis of the crystalline surface and reddish color at an angle far away from the normal axis.

(3) For particles with sizes of approximately 410 nm, the hair fibres showed a strong greenish diffraction colour at a viewing angle close to normal axis of the crystalline surface and reddish colour at an angle far away from the normal axis.

(4) For particles with sizes of approximately 500 nm, the hair fibres showed a strong reddish diffraction colour.

Each test was performed on hair from 3 different models. The cosmetic characteristics of the hairstyles were then evaluated by a panel of 3 individuals. The test samples of hair were in the form of a comb of 10-30 fragments of hair 75 mm long bonded in parallel onto a plastic support.

To demonstrate the particle deposition on the fibre by applying the dispersion, optical measurement after before treatment, followed by a second measurement after treatment. The measurements and also the drying of the test samples of hair were performed at controlled temperature and relative humidity (20° C. and 45% relative humidity).

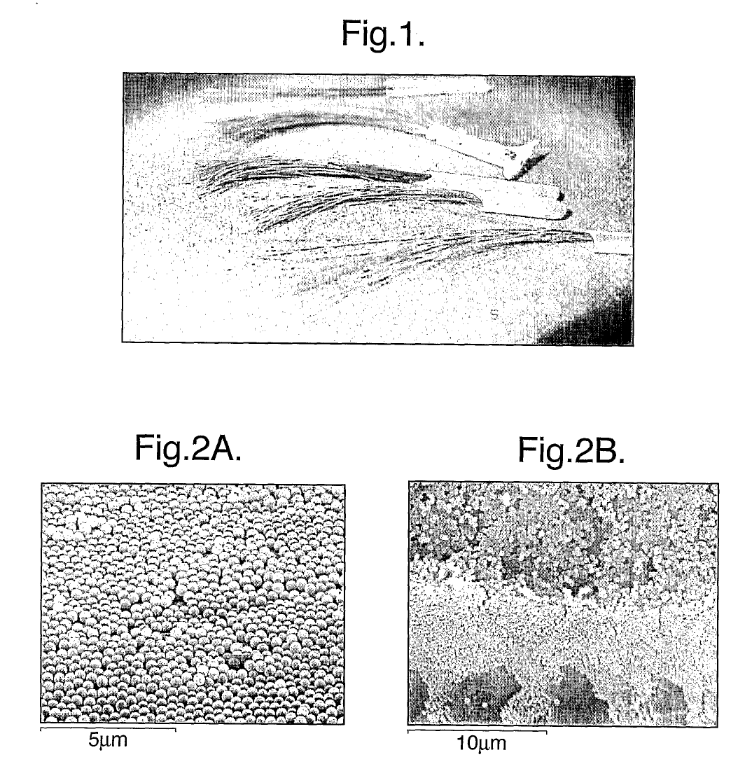

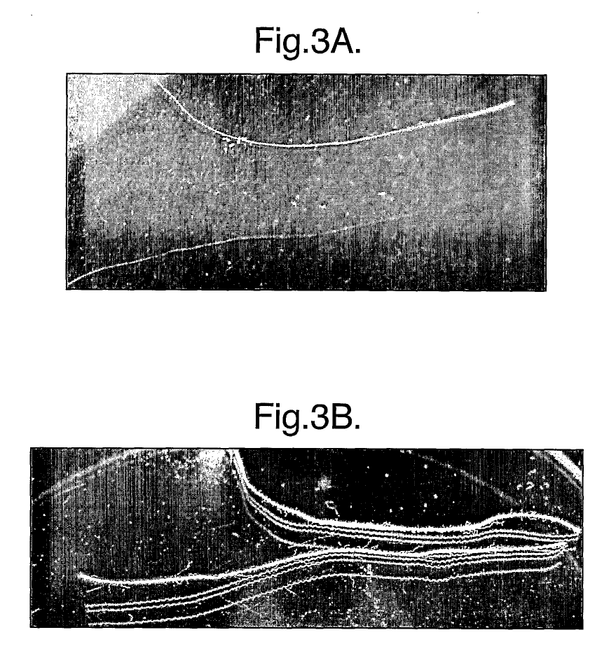

Examples of the crystalline hair fibres are shown in FIG. 1. From top to bottom in this image, the fibre bundles were coloured, respectively, bronze, indigo, blue, green and pink. Examples of the formation of crystalline layers on hair are shown by SEM-measurements in FIG. 2.

### Discussion

We have shown that highly monodisperse SiO₂-particles assemble via a self-organisation process into three dimensional crystal lattices on hair fibres. Well defined structured particle layers were generated using a modified vertical deposition method. The hair substrate is immersed vertically into a suspension containing the monodisperse spheres. The crystal grows continuously on the hair surface, since the liquid level decreases via evaporation. During the evaporation of the solvent, the surface of the solvent moves downwards and the silica particles deposit onto the substrate since the remaining liquid film on the hair becomes thinner than the actual particle diameter.

The thickness of the crystals is controlled by the concentration of the colloids and the shape of the meniscus at the hair surface. Desiccation speed has little influence. The shape of the meniscus depends on the dewetting-qualities of the liquid phase on the hair substrate. Aqueous suspensions (high surface tension) lead to thicker crystals, but with a decrease in quality.

The characterised crystals reveal a fcc-lattice of hexagonal close packed particles with some local defects and grain boundaries, where the top lattice corresponds to the (111)-surface.

In this method the concentration of particles changes during solvent evaporation, which may have an effect on the film thickness.

Nevertheless, the visual appearance of the films testifies to their high crystalline quality and their uniform thickness. The samples exhibit a brilliant colour due to Bragg diffraction of visible light. A systematic change of colour can be seen by modifying the orientation of the substrate.

In controlled drying techniques, templates can direct colloidal crystallisation. The ability to form such a templated crystal is likely to be dependent on the surface topography of the template. We believe the irregular surface texture of the hair-fibre acts as nucleation sites for crystal growth.

### Example 2

### Assembly of Colloidal Crystals on Cotton Fibres

### Synthesis of Silica-Particles

Essentially as per example 1.

### Crystal Growth on Cotton Substrate

The sample consisted of a silica concentration of about 0.2 weight % in anhydrous ethanol. Cotton yarns were placed vertically into each dispersion. The dispersion in the container was left to evaporate/crystallize at room temperature overnight.

For particles with sizes of approximately 250 nm, the cotton yarns showed strong blueish diffraction at a viewing angle close to the normal axis of the crystalline surface on the cotton yarns.

To demonstrate the particle deposition on the fibre by applying the dispersion, optical measurement after before treatment, followed by a second measurement after treatment. The measurements and also the drying of the test samples of cotton were performed at controlled temperature and relative humidity (20° C. and 45% RH).

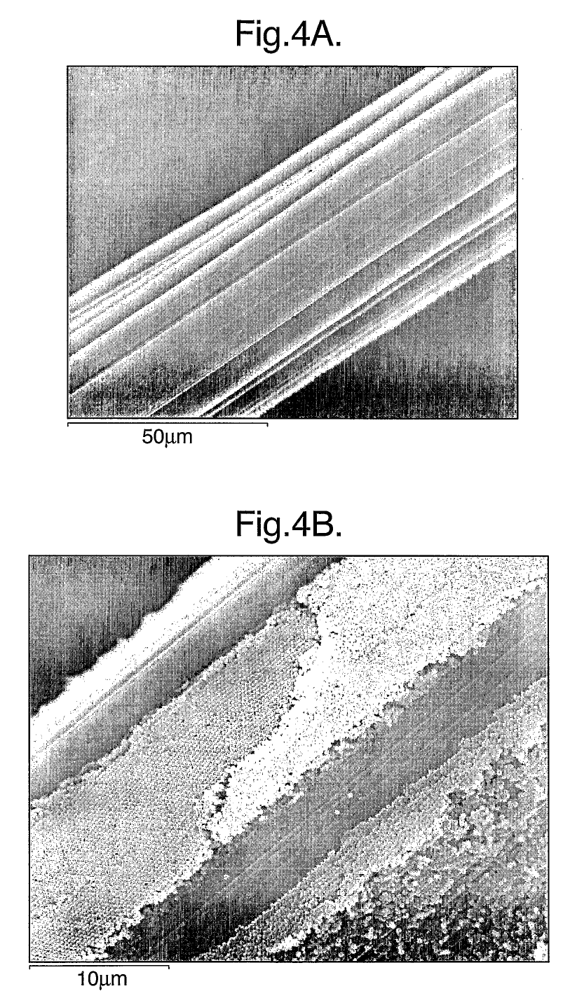

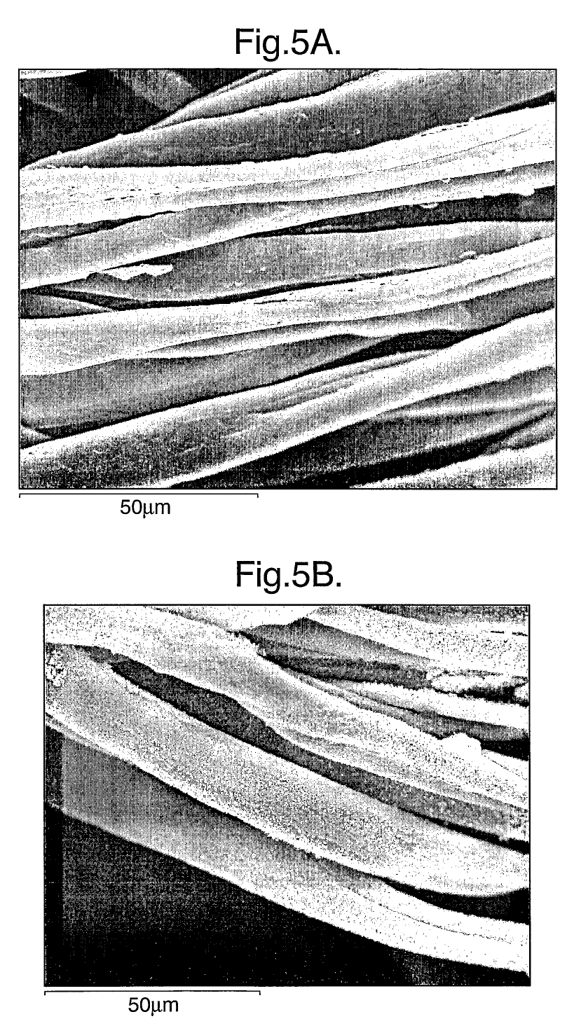

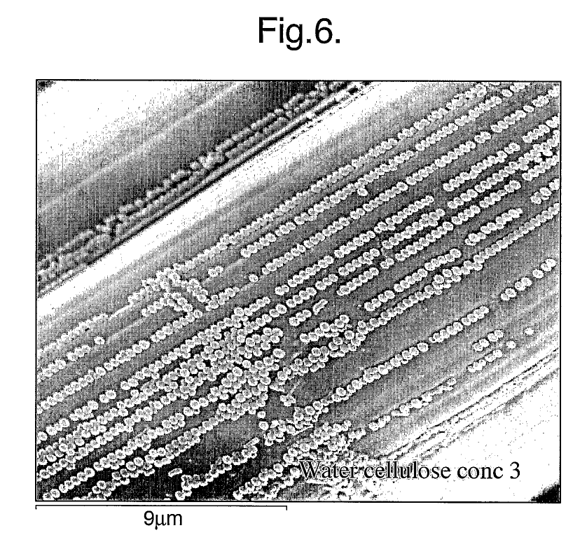

Examples of the crystalline cotton samples are shown in FIG. 3. Examples of the formation of crystalline layers on cotton are shown by SEM-measurements in FIGS. 4 and 5. Again growth under controlled conditions indicated that surface topography is important for templating crystal growth (see FIG. 6 where crystal growth is initiated along the surface ridges of the cellulose fibre).

The various features and embodiments of the present invention, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and products of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the relevant fields are intended to be within the scope of the following claims.

1. A method of colouring a substrate selected from hair of an individual and fabric fibres, which method comprises contacting the substrate with a composition comprising monodisperse spherical particles capable of forming a colloidal crystal that diffracts light having a wavelength in a range that corresponds to the wavelength of visible light, such that colloidal crystals comprising the monodisperse particles form on the substrate; wherein the colloidal crystal has a lattice spacing in a range that corresponds to the wavelength of visible light.

2. A method according to claim 1, wherein the substrate is the hair of an individual and at least a region of the hair is contacted with the composition such that the monodisperse particle form on at least a portion of the hair.

3. A method of printing onto a substrate which method comprises contacting at least a region of the substrate with an ink composition comprising monodisperse spherical particles capable of forming a colloidal crystal having a lattice spacing in a range that corresponds to the wavelength of visible light, such that colloidal crystals comprising the monodisperse particles form on at least a portion of the substrate; wherein the colloidal crystal has a lattice spacing in a range that corresponds to the wavelength of visible light.

4. (canceled)

5. (canceled)

6. (canceled)

7. (canceled)

8. A method according to any claims 1 or 3, wherein the colloidal crystals have a lattice spacing in at least one axis of about 380 nm to about 770 nm.

9. A method according to claim 1 or 3, wherein the monodisperse particles deviate less than 10% in rms diameter.

10. A method according to claim 1 or 3, wherein the monodisperse particles have an rms diameter of about 200 nm to about 500 nm.

11. A method according to claim 1 or 3, wherein the monodisperse particles are formed from one or more materials selected from organic and inorganic materials, selected from latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate), metal chalcogenide, metal pnictide, silica, metal and metal oxide particles. for example of Al₂O₃, TiO₂, SnO₂, Sb₂O₅, Fe₂O₃, ZrO₂, CeO₂ and Y₂O₃, gold, copper and silver, and mixtures of any one or more of any of the foregoing.

12. (canceled)

13. (canceled)

14. (canceled)

15. A fibrous material comprising at least one colloidal crystalline layer comprising monodisperse particles, which layer diffracts light having a wavelength in a range that corresponds to the wavelength of visible light.

16. A fibrous material according to claim 15, which is a fabric.

17. (canceled)

18. A fibre colourant or ink composition comprising monodisperse particles capable of forming a colloidal crystal that diffracts light having a wavelength in a range that corresponds to the wavelength of visible light.

19. (canceled)
